(19) United States
(12) Patent Application Publication
(10) Pub. No.: US 2026/0063628 A1
(43) Pub. Date: Mar. 5, 2026

(54) METHOD OF DETECTING TARGET SUBSTANCE AND DETECTION REAGENT FOR TARGET SUBSTANCE

(71) Applicant: CANON KABUSHIKI KAISHA, Tokyo (JP)

(72) Inventors: FUMIO YAMAUCHI, Kanagawa (JP); NORISHIGE KAKEGAWA, Tokyo (JP)

(21) Appl. No.: 19/317,740

(22) Filed: Sep. 3, 2025

(30) Foreign Application Priority Data
Sep. 4, 2024 (JP) 2024-152067

(51) Int. Cl. G01N 33/543; G01N 1/38; G01N 21/64; G01N 21/82; G01N 33/545; G01N 33/552

(52) U.S. Cl. CPC G01N 33/54313; G01N 1/38; G01N 21/6445; G01N 21/82; G01N 33/545; G01N 33/552; G01N 2021/825

(57) ABSTRACT

To provide a method of detecting a target substance in which silica particles are used, and a sensitizing factor is increased without any reduction in measurement accuracy, provided is a method of detecting a target substance including: a first step of mixing a specimen liquid containing at least the target substance and a particle dispersion, which comprises first particles each having a site that specifically binds to the target substance, to produce a liquid sample; and a second step of performing the optical measurement of the liquid sample, wherein the liquid sample comprises second particles, wherein the ratio of the average particle diameter of the second particles to the average particle diameter of the first particles in the liquid sample is 0.03 or more and 0.24 or less, and wherein the refractive index of the second particles is smaller than the refractive index of the first particles.

### BACKGROUND

### Field of the Technology

The present disclosure relates to a method of detecting a target substance and a detection reagent for a target substance.

### Description of the Related Art

An immunoassay method utilizing an antigen-antibody reaction is currently used in a clinical examination for detecting and measuring a target substance in a specimen. Of those, an immunoassay method including using particles each having a site that specifically binds to a target substance, such as an antibody or an antigen, is known.

A latex agglutination method has been used as such an immunoassay method. In an example of the latex agglutination method, a target substance can be qualitatively and quantitatively measured as follows: a specimen (e.g., a serum) that may contain the target substance is caused to react with a latex agglutination reagent containing latex particles (also referred to as “antibody-sensitized particles”) each having bonded thereto an antibody against the target substance; and the agglutination of an aggregate produced by the antigen-antibody reaction is visually or optically detected.

In addition, as another immunoassay method, U.S. Patent Application Publication No. 2024/0093087 provides a fluorescence polarization immunoassay method including using luminescent particles. In an example of the fluorescence polarization immunoassay method, a target substance can be qualitatively and quantitatively measured as follows: a specimen that may contain the target substance is caused to react with a luminescent reagent containing luminescent particles (also referred to as “antibody-sensitized luminescent particles”) each having bonded thereto an antibody against the target substance; and the agglutination of the aggregate of the luminescent particles produced by the antigen-antibody reaction is detected by a fluorescence polarization method.

A detection reagent using particles, such as latex particles or luminescent particles, is generally used as an aqueous dispersion obtained by dispersing the particles in an aqueous solvent.

Although a high-sensitivity chemiluminescent immunoassay method has also been used in a clinical examination, its examination time tends to be longer because the method requires bound/free (BF) separation (step of removing an unreacted component). Meanwhile, in each of the latex agglutination method and the fluorescence polarization immunoassay method that do not require the BF separation, an examination time is shorter and an examination throughput is higher. However, detection sensitivity may be insufficient when compared to the chemiluminescent immunoassay method, and hence a further improvement in detection sensitivity has been required.

A water-soluble polymer has been used as a sensitizer for the purpose of improving the sensitivity of the latex agglutination method or the fluorescence polarization immunoassay method.

In addition, Japanese Patent Laid-Open No. 2019-211449 provides a reagent for measuring a complement value containing a micrometer-sized sensitized erythrocyte or sensitized liposome suspended in a buffer, the reagent for measuring a complement value containing silica particles. Japanese Patent Laid-Open No. 2019-211449 discloses that when the silica particles are mixed as a measured value stabilizer into the reagent for measuring a complement value, the erythrocyte in the reagent does not sediment, and hence the complement value can be accurately measured even when the reagent is not stirred immediately before the measurement.

In addition, Japanese Patent Laid-Open No. H11-337551 provides a nonspecific reaction inhibitor to be used in a detection method utilizing particle agglutination in which latex particles each having immobilized thereon an antibody or an antigen against a substance to be measured are used, the inhibitor being formed of particles each having immobilized thereon an antibody, which does not immunologically react with the above-mentioned substance to be measured, or a fragment thereof, or an antigen that does not immunologically react therewith; and an immunoassay using the inhibitor.

Japanese Patent Laid-Open No. H11-337551 provides particles each having bonded thereto an immunologically inert protein function as a nonspecific reaction inhibitor during a latex agglutination reaction using the latex particles each having bonded thereto the antibody or the antigen.

### SUMMARY

To solve the above-mentioned problems, according to one embodiment of the present disclosure, there is provided a method of detecting a target substance including: a first step of mixing a specimen liquid containing at least the target substance and a particle dispersion, which comprises first particles each having a site that specifically binds to the target substance, to produce a liquid sample; and a second step of performing optical measurement of the liquid sample, wherein the liquid sample comprises second particles, wherein a ratio of an average particle diameter of the second particles to an average particle diameter of the first particles in the liquid sample is 0.03 or more and 0.24 or less, and wherein a refractive index of the second particles is smaller than a refractive index of the first particles.

In addition, according to one embodiment of the present disclosure, there is provided a detection reagent for a target substance including at least: first particles each having a site that specifically binds to the target substance; and second particles that are different in kind from the first particles, wherein a ratio of an average particle diameter of the second particles to an average particle diameter of the first particles is 0.03 or more and 0.24 or less, and wherein a refractive index of the second particles is smaller than a refractive index of the first particles.

Features of the present disclosure will become apparent from the following description of embodiments. The following description of embodiments is described by way of example.

### DESCRIPTION OF THE EMBODIMENTS

A detection reagent according to one embodiment of the present disclosure, which includes at least: first particles each having a site that specifically reacts with a target substance; and second particles that are each free of any site that specifically reacts with the target substance, and a method of detecting a target substance in a specimen with the reagent are described below. However, the present disclosure is not limited thereto.

### First Embodiment

A detection reagent for a target substance of a first embodiment is a detection reagent for a target substance including at least: first particles each having a site that specifically reacts with the target substance; and second particles that are different in kind from first particles, wherein a ratio of an average particle diameter of second particles to the average particle diameter of the first particles is 0.03 or more and 0.24 or less, and wherein a refractive index of the second particles is smaller than a refractive index of the first particles.

A search has been implemented for a substance that accelerates the agglutination reaction of the first particles each having a site that specifically reacts with the target substance for the purpose of establishing a high-sensitivity and high-accuracy detection method. As a result, it has been found that the second particles each having a low refractive index, second particles having an average particle diameter smaller than the average particle diameter of the first particles, achieve both of an improvement in sensitivity of an immunoassay by the first particles each having a site that specifically reacts with the target substance, and prevention of a reduction in measurement accuracy. In addition, it has been found that the second particles in a reagent have a suppressing effect on the spontaneous sedimentation of the first particles present in a same reagent, the particles each having a site that specifically reacts with the target substance.

The detection reagent for a target substance of this embodiment is a detection reagent for a target substance to be used in, for example, the immunoassay of the target substance in a specimen, the immunoassay using the first particles each having a site that specifically reacts with the target substance. The term “immunoassay method” as used herein refers to an immunoassay method based on an antigen-antibody reaction, and includes an immunoassay method called, for example, latex agglutination immunonephelometry, a latex agglutination method, or a fluorescence polarization immunoassay method. A specific example thereof is a method of measuring an antigen with particles each having bonded thereto an antibody. The detection reagent for a target substance of this embodiment detects the target substance through use of a latex agglutination method or a fluorescence polarization immunoassay method. The present disclosure is described below.

### (Target Substance in Specimen)

The detection reagent of this embodiment is used in a measurement of a target substance in a liquid to be examined (the liquid may be a specimen liquid, or may be a liquid sample obtained by mixing a specimen liquid to be subjected to optical measurement and a reagent), which may contain the target substance, in the detection method of this embodiment.

The target substance only needs to be one of specific binding partners, and is one of binding partners, such as a ligand and an acceptor, a ligand and a receptor, or an antigen and an antibody. The substance is, for example, a substance that can be measured by an immune reaction, and examples thereof include substances, such as an antigen and an antibody. The term “specimen liquid” as used herein means a liquid to be examined that may contain the target substance, and the liquid may be a liquid before its mixing with a reagent, or may be a sample liquid mixed with a reagent to be subjected to optical measurement.

Examples of the target substance include C-reactive protein (CRP), ferritin, albumin, immunoglobulin G (IgG), immunoglobulin A (IgA), immunoglobulin M (IgM), immunoglobulin E (IgE), α-fetoprotein (AFP), carcinoembryonic antigen (CEA), prostate specific antigen (PSA), KL-6, pepsinogen (PG), rheumatoid factor (RF), lipoprotein, insulin, an infection-related antigen and an antibody against the antigen, a hormone, a vitamin, and an antibiotic. The examples of the target substance also include a nucleic acid, an enzyme, an enzyme substrate, a virus, and an extracellular vesicle.

(First Particles Each Having Site that Specifically Binds to Target Substance)

The detection reagent for a target substance of this embodiment includes at least: the first particles each having a site that specifically binds to the target substance; and the second particles that are different in kind from the first particles. The first particles that may be used in the detection reagent of this embodiment are particles each having a site that specifically binds to the target substance.

The term “first particles” as used herein is identical in meaning to an insoluble carrier, and means a particulate carrier that is substantially insoluble in water. Accordingly, the first particles may also be referred to as “insoluble carrier particles.” The shapes of the first particles are not particularly limited as long as the first particles can be dispersed in water, and hence can be subjected to optical measurement, such as turbidity measurement or fluorescence polarization measurement. A material for the first particles is, for example, a resin such as polystyrene, a natural polymer, such as agarose or dextran, an inorganic substance such as alumina, or a metal, such as iron oxide or gold colloid.

The synthesis and preparation of the first particles may be performed in accordance with a known method. Although any particles to be used in a typical latex agglutination reaction or fluorescence polarization immunoassay method may be used as the first particles, particles close to true spheres are useful. The first particles to be used in the fluorescence polarization immunoassay method each emit polarized light that can be detected in the immunoassay, and the particles are, for example, luminescent material-containing particles.

The first particles are suitable, for example, latex particles or luminescent particles. The term “latex particles” as used herein means polymer particles on each of which an antigen or an antibody against the target substance can be immobilized, the particles being used in a latex agglutination reaction. The latex particles can be, for example, polystyrene particles. The sizes of the polystyrene particles can be controlled, and hence particles having a narrow size distribution (particle size distribution) are obtained. In addition, the polystyrene particles are useful in terms of cost because a method of synthesizing a large amount of the particles has been established.

The term “luminescent particles” as used herein means light-emitting particles on each of which an antigen or an antibody against the target substance can be immobilized, the particles being used in, for example, a fluorescence polarization method. Those particles are carriers that may be suitably used with the detection reagent of this embodiment because the particles have, for example, the following advantages: nanosized particles are obtained with relative ease; and surfaces of the particles can be chemically modified in accordance with purposes. The luminescent particles can be particles obtained by incorporating luminescent molecules into polystyrene particles. The luminescent molecules only need to be molecules each emitting fluorescence, and can be molecules each emitting polarized fluorescence. The luminescent molecules are can each be a rare earth complex.

The first particles each have a site that specifically binds to the target substance. Herein, the site that specifically binds to the target substance can be a site that can specifically bind and adsorb to the target substance. The site that specifically binds to the target substance is, for example, an antibody or an antigen. The antibody may be a monoclonal antibody or a polyclonal antibody, or a fragmented antibody (also referred to as “antibody fragment”), such as Fab, F(ab′)2, F(ab′), Fv, or scFv, may be used. In addition to the foregoing, an appropriate substance may be selected in accordance with the target substance. Examples thereof include: an enzyme and a substrate thereof; a signal substance, such as a hormone or a neurotransmitter, and a receptor thereof; a nucleic acid; avidin; and biotin.

The first particles each have a site that specifically binds to the target substance. The specific reaction is also referred to as “sensitization”, “adsorption”, or “immobilization”. Although a site that specifically binds to the target substance may be immobilized on a base particle through a chemical bond, or may be immobilized thereon by physical adsorption, the site can be immobilized thereon through a chemical bond. Although the immobilization of the site that specifically binds to the target substance only needs to be performed in accordance with an ordinary method, a functional group on a surface of the base particle may be caused to react with, for example, a primary amine, a secondary amine, a carboxyl group, or a thiol group having a site that specifically binds to the target substance.

Examples of the functional group of the base particle to which the site that specifically binds to the target substance is bonded include a carboxyl group, an amino group, an aldehyde group, an epoxy group, a thiol group, and a malcimide group. The particle may have one kind of those functional groups or two or more kinds thereof.

With regard to the site that specifically binds to the target substance, the site which is bonded to the base particle, the presence or absence of the binding, and the binding amount of the substance may be determined by using a known method of detecting the site that specifically binds to the target substance. For example, when the site that specifically binds to the target substance is a protein, an amount of the protein bonded to the first particles may be determined by, for example, a BCA assay by which the amount of the protein is determined.

In the detection reagent for a target substance of this embodiment, an average particle diameter of the first particles can be 50 nm or more and 500 nm or less, or 100 nm or more and 400 nm or less in terms of major axis diameter. When the average particle diameter of the first particles is set to 50 nm or more, the absorbance of the reagent in a visible range to be observed in latex agglutination measurement falls within a moderate range. In addition, when the average particle diameter of the first particles is set to 50 nm or more, a large amount of a luminescent material can be incorporated into the first particles, and hence particles each having a luminescence intensity enough for observation by fluorescence polarization measurement can be obtained.

When the average particle diameter of the first particles is set to 500 nm or less, the dispersion stability of the first particles in a solution is improved, and an absorbance in the visible range falls within a moderate range. From viewpoints of reactivity and dispersion stability, particles having an average particle diameter of 100 nm or more and 400 nm or less can be used. The average particle diameter may be represented by a z-average particle diameter measured by particle diameter measurement based on a dynamic light scattering method. Herein, the average particle diameter of the first particles is the average particle diameter of particles each having a site that specifically binds to the target substance.

In addition, the concentration of the first particles may be appropriately adjusted by the target substance to be measured and the material for the first particles in accordance with the average particle diameter and specific gravity thereof, and with conditions for optical measurement. With regard to the detection reagent for a target substance of the present disclosure, the concentration of the first particles in the detection reagent can be 0.0002 mass % or more and 2.0 mass % or less from viewpoints of reactivity and a solution turbidity. Specifically, in a latex agglutination method by which the turbidity of the reagent in a visible range is measured, the concentration of the first particles in the reagent can be 0.01 mass % or more and 2.0 mass % or less. When the concentration falls within the range, the turbidity in the visible range can be reliably detected, and the measurement range of the turbidity (absorbance) can be sufficiently secured.

The concentration of the first particles can be at most 2.0 mass % so that the turbidity (absorbance) may not exceed a measurement upper limit. In addition, when luminescence measurement is performed like a fluorescence polarization method including using luminescent particles, the concentration of the first particles in the reagent can be even lower. This is because the scattering of the first particles inhibits the luminescence measurement. Specifically, the concentration of the luminescent particles in the reagent can be 0.0002 mass % or more and 0.01 mass % or less. When the concentration falls within the range, the measurement of light emitted from the luminescent particles stabilizes.

The concentration of particles in a liquid sample is the solid content concentration of the particles in a dispersion (hereinafter also referred to as “particle dispersion”), and may be measured with, for example, a heat-drying moisture meter (e.g., a heat-drying moisture meter MX-50).

In this embodiment and Examples to be described later, the true specific gravity (sometimes abbreviated as “specific gravity”) of particles was determined by measuring density of the particles. The true specific gravity of the particles is the relative density thereof with respect to water, and density of water is set to 1 g/cm³. First, a particle dispersion was freeze-dried, and the density of its particles was determined by a dry density measurement method. A measuring apparatus that performs the dry density measurement is, for example, a dry automatic density-measuring instrument “Accupyc II” manufactured by Shimadzu Corporation. A value obtained by dividing the density thus determined by the density of water was adopted as the true specific gravity of the particles. As described later in Examples, for example, the following values were obtained: specific gravity of polystyrene particles was 1.05, and specific gravity of silica particles was 1.8.

(Second Particles that are Each Free of Site that Specifically Binds to Target Substance)

The detection reagent for a target substance of this embodiment includes at least: the first particles each having a site that specifically binds to the target substance; and the second particles that are different in kind from the first particles. In the detection reagent for a target substance of this embodiment, the second particles can be particles that are each free of any site that specifically binds to the target substance. In this embodiment, a component, which is responsible for an improvement in sensitivity of the method of detecting a target substance and the stabilization of the detection reagent including the first particles each having a site that specifically binds to the target substance, is the second particles that are each free of any site that specifically binds to the target substance. The second particles have the following features: unlike the first particles, the second particles are each free of any site that specifically binds to the target substance; their average particle diameter is smaller than that of the first particles; the concentration of the particles is higher than that of the first particles; and their refractive index is lower than that of the first particles.

In this embodiment, refractive index measurement may be performed by a known method. For example, an immersion method or a method including using Lorentz-Lorenz equation has been known, and may be appropriately selected.

In the immersion method, light scattering measurement is performed by changing the refractive index of a dispersion. In the method, the following fact is utilized: when the refractive indices of the dispersion and its particles coincide with each other, an apparently transparent solution is obtained, and hence a light scattering property reduces. The refractive index when the light scattering property disappears is the refractive index of the particles.

The refractive indices may be determined by using the Lorentz-Lorenz equation. In the method, first, the refractive index of a particle dispersion is determined with a refractometer for a liquid. Next, the refractive index of its particles (solid content) is calculated by using the Lorentz-Lorenz equation. Details are described below.

In the method, the solid content concentration of the particle dispersion is required in advance. For example, the solid content concentration may be measured with a heat-drying moisture meter (e.g., a heat-drying moisture meter MX-50). The density of the particles is also required, and the density may be determined as follows: the dispersion is freeze-dried, and its density is measured with a dry automatic density-measuring apparatus.

First, the refractive index of the particle dispersion is measured. An Abbe refractometer (e.g., a multiwavelength-type digital Abbe refractometer Abbemat MW) may be used as the refractometer for a liquid. Herein, a measurement wavelength of 589.3 nm and a measurement temperature of 23° C. are typically selected. Next, the refractive index of the particles is calculated. The refractive index of a mixture depends on the volume fractions of its constituents. The particle dispersion is regarded as the two-component system of the particles and water, and their volume fractions are calculated from their concentrations and densities. Then, the refractive index of the particles may be calculated by using the Lorentz-Lorenz equation. Herein, the following values may be used: specific gravity and refractive index of water are 1.00 and 1.333, respectively.

As described later in Examples, the following values were obtained as examples of the results of the measurement method including using the Lorentz-Lorenz equation: the refractive index of polystyrene particles was 1.59, and the refractive index of silica particles was 1.45.

In this embodiment, a fact that the second particles are silica particles may be recognized by a known analysis method.

For example, silicon nuclear magnetic resonance spectrometry (NMR) or high-frequency inductively coupled plasma (ICP)-atomic emission spectroscopy is applicable. The second particles and the first particles may be separated from each other by centrifugal separation or ultrafiltration through utilization of a difference in size from the first particles, and may each be subjected to component analysis.

In this embodiment, the second particles can be particles that are each free of any site that specifically binds to the target substance, and the fact that the particles are each free of any site that specifically binds to the target substance may be recognized by a known analysis method. For example, the following may be performed: the second particles and the first particles are separated from each other by applying nuclear magnetic resonance spectrometry (NMR) or high-frequency inductively coupled plasma (ICP)-atomic emission spectroscopy; and protein quantification and immunoassay of the site that specifically binds to the target substance are performed on the second particles. For example, when the site that specifically binds to the target substance is a protein, a fact that the site that specifically binds to the target substance is not bonded to any one of the second particles may be recognized by using a commercial protein stain solution (e.g., a Coomassie stain solution) for the second particles.

The action mechanism of the second particles is described below. In the method of detecting a target substance according to this embodiment, the mechanism via which the second particles can improve sensitivity of detection of the target substance by the first particles each having a site that specifically binds to the target substance may result from an apparent increase in optical path length (increase in absorbance) due to multiple scattering and a depletion agglutination action, which are caused by presence of the second particles.

The term “depletion agglutination” as used herein refers to agglutination of large particles caused as follows: in the case where the large particles and many small particles are present in a solution (in this embodiment, the small particles may be the “second particles that are each free of any site that specifically binds to the target substance,” and the large particles may be the “first particles each having a site that specifically binds to the target substance”), when the large particles approach each other, the small particles are excluded from a gap between the large particles, and as a result, an osmotic pressure acts between the large particles to cause the agglutination. That is, to effectively cause the depletion agglutination of the large particles, it is effective to use small particles, which weakly interact with various media in an aqueous solution and have an appropriate average particle diameter.

In this embodiment, the second particles weakly interact with a salt and an ion, and also weakly interact with a protein and the first particles each having a site that specifically binds to the target substance. In addition, the following fact has been found for a first time in the present disclosure: the second particles do not significantly increase viscosity of a solution, and the refractive index of the second particles is low, and hence their scattering properties in an aqueous solution are low; accordingly, the particles are extremely effective as an additive (sensitizer) for a detection reagent utilizing optical measurement, the reagent including the first particles each having a site that specifically binds to the target substance.

In the detection reagent for a target substance of this embodiment, the second particles can serve as a sensitizer in the detection of the target substance and as a dispersant for the first particles. In the reagent of this embodiment, mechanism via which when the second particles are each free of any site that specifically binds to the target substance, the particles stabilize the first particles each having a site that specifically binds to the target substance (the second particles function as a dispersant or stabilizer for the first particles) may result from a thickening effect on a reagent solution and an increasing effect on the specific gravity of the reagent solution.

That is, the spontaneous sedimentation of the first particles each having a site that specifically binds to the target substance is suppressed by thickening of the reagent solution and an increase in specific gravity thereof. In addition, the second particles that are each free of any site that specifically binds to the target substance may have a physically inhibiting effect on an interaction between the first particles each having a site that specifically binds to the target substance.

The phrase “free of any site that specifically binds to the target substance” as used herein refers to a state in which the particles are each completely free of any site that specifically binds to the target substance, or even when the particles each have a site that specifically binds to the target substance, the particles are each free of any function of binding thereto (when the target substance is an antigen, an ability to bind to the antigen, or when the target substance is an antibody, an ability to bind to the antibody). The foregoing is represented in terms of, for example, the binding amount of the antigen or the antibody with respect to the mass of carriers (base particles) as follows: the amount of the site that specifically binds to the target substance is 0.1 μg or less with respect to 1 mg of the carriers.

In order that the second particles may express the above-mentioned sensitivity-improving effect, the second particles are required to interact with various substances in a reaction liquid including the target substance in an extremely weak manner. Accordingly, such second particles are essentially different from the nonspecific reaction inhibitor disclosed in Japanese Patent Laid-Open No. H11-337551 in a sense that the particles do not capture anything.

As in the first particles, the term “second particles” as used herein means a particulate carrier that is substantially insoluble in water. The shapes of the particles are not particularly limited as long as the particles are dispersed in water, and hence do not inhibit optical measurement, such as turbidity measurement or fluorescence polarization measurement. A material for the particles is, for example, low-refractive index particles, such as amorphous fluorine polymer, polymethyl methacrylate, or silica particles.

In the detection reagent for a target substance of this embodiment, a refractive index of the second particles can be smaller than a refractive index of the first particles. This is because when the refractive index of the second particles is lower than the refractive index of the first particles, scattering properties from the second particles reduce, and hence no longer inhibit turbidity measurement or fluorescence polarization measurement during the detection of the target substance with the first particles. That is, the first particles can be particles having a relatively high refractive index such as polystyrene. The refractive index of the polystyrene is 1.59. In the detection reagent for a target substance of this embodiment, the refractive index of the second particles can be less than 1.59, or 1.50 or less, or 1.48 or less. That is, in the detection reagent for a target substance of this embodiment, a ratio of the refractive index of the second particles to the refractive index of the first particles can be 0.92 or less.

The synthesis and preparation of the second particles may be performed in accordance with a known method. The particles can be particles close to true spheres. The second particles are suitably, for example, silica particles. This is because of the following reasons: the silica particles have a refractive index as low as 1.50 or less; particles close to true spheres can be easily synthesized by a known method; the particles can be controlled to have various average particle diameters and surface states; and the particles can be produced in a large amount at low cost.

In the detection reagent for a target substance of this embodiment, the true specific gravity of the second particles can be larger than the true specific gravity of the first particles. This is because of the following reason: the second particles having a relatively large specific gravity contribute to an increase in specific gravity of a reagent solution; accordingly, when the second particles coexist with the first particles, the spontaneous sedimentation of the first particles each having a site that specifically binds to the target substance can be suppressed. The true specific gravity of the second particles is 1.1 or more and 3.0 or less, or 1.5 or more and 2.2 or less because the second particles sediment when their specific gravity is excessively large.

Surfaces of the second particles can each be free of any site that specifically binds to the target substance. Meanwhile, to suppress an interaction with any other substance, the surfaces can be hydrophilic surfaces to each of which no protein adsorbs. For example, the surfaces of the second particles can each be coated with a hydrophilic polymer. Although the hydrophilic polymer is not particularly limited as long as the hydrophilic polymer has low protein adsorptivity, examples thereof include polyvinylpyrrolidone (PVP) and polyethylene glycol.

When the second particles are each coated with the hydrophilic polymer, their interactions with contaminants (e.g., a protein except the target substance) in a specimen can be suppressed. An interaction between the hydrophilic polymer and a surface of the base of each of the second particles for the coating with the hydrophilic polymer may be a noncovalent bond between the hydrophilic polymer and the surface of the base of the second particle, such as a hydrogen bond, an ionic bond, or a van der Waals bond, or may be a covalent bond between a synthetic polymer and the second particle. The term “base of each of the second particles” as used herein refers to a base in a state before the second particle is obtained by the surface coating.

When the hydrophilic polymer is bonded to the surface of the base of each of the second particles through a covalent bond, for example, the functional groups of the base of the particle and the hydrophilic polymer may be utilized. Examples of the functional groups include a primary amine, a secondary amine, a carboxyl group, a thiol group, an aldehyde group, an epoxy group, and a maleimide group. With regard to the surface of each of the second particles and the hydrophilic synthetic polymer present on the surface, the composition of the surface, presence or absence of a functional group, presence or absence of the polymer, the molecular weight of the polymer, the composition of the polymer, and an amount of the polymer with which the surface is coated may be determined by using known analysis methods.

The average particle diameter of the second particles is smaller than that of the first particles. The average particle diameter may be represented by, for example, a z-average particle diameter measured by particle diameter measurement based on a dynamic light scattering method. In the detection reagent for a target substance of this embodiment, the average particle diameter of the second particles can be 5 nm or more and 55 nm or less, or 10 nm or more and 50 nm or less, or 10 nm or more and 30 nm or less in terms of major axis diameter.

When the average particle diameter of the second particles is set to 5 nm or more, it is conceivable that the particles show an effective average particle diameter in an aqueous medium to facilitate the expression of the depletion agglutination effect of the first particles, and hence the measurement sensitivity of the agglutination reaction of the particles can be further improved. In addition, the average particle diameter of the second particles can be set to 5 nm or more because the preparation work of the second particles including centrifugal separation work becomes easier.

In addition, when the average particle diameter of the second particles is set to 55 nm or less, the scattering of light in a visible range can be suppressed. The average particle diameter of the second particles can be set to 55 nm or less because the scattering components of the second particles themselves may hardly affect measurement of a signal (absorbance or light emission) of optical measurement.

In this embodiment, a size ratio between the first particles and the second particles is important. In other words, in the detection reagent for a target substance of this embodiment, the ratio of the average particle diameter of the second particles to the average particle diameter of the first particles is 0.03 or more and 0.24 or less. When the ratio of the average particle diameter of the second particles to the average particle diameter of the first particles is 0.03 or more, as described above, it is conceivable that the second particles show an effective size in an aqueous medium to enable the expression of the depletion agglutination effect of the first particles, and hence the measurement sensitivity of the agglutination reaction of the particles can be improved.

Meanwhile, when the ratio of the average particle diameter of the second particles to the average particle diameter of the first particles becomes excessively large, for example, when the second particles are identical in size to the first particles (the ratio between the average particle diameters is 1), a state in which the depletion agglutination occurs is no longer established, and hence the agglutination reaction of the first particles is inhibited in some cases. In addition, when the ratio of the average particle diameter of the second particles to the average particle diameter of the first particles becomes excessively large, light scattering from the second particles significantly affects the optical measurement of the first particles in some cases.

As clarified in Examples to be described later, when the ratio of the average particle diameter of the second particles to the average particle diameter of the first particles is 0.24 or less, as described above, the depletion agglutination of the first particles easily occurs and the influence of the light scattering from the second particles can be reduced. Accordingly, in the reagent of this embodiment, the ratio of the average particle diameter of the second particles to the average particle diameter of the first particles can be adjusted to 0.03 or more and 0.24 or less.

Further, from a viewpoint that the scattering by the second particles is wished to be further reduced, the second particles can be as small as possible. Accordingly, the ratio between the average particle diameters can be 0.03 or more and 0.12 or less. From a viewpoint of the achievement of both the improvement in sensitivity and the maintenance of measurement accuracy (satisfactory reproducibility) by the second particles, a ratio between the average particle diameters can be 0.04 or more and 0.10 or less.

In the detection reagent for a target substance of this embodiment, concentration of the second particles is an important parameter, and a ratio of the concentration (mass basis, mass %) of the second particles to the concentration (mass basis, mass %) of the first particles in a liquid sample can be 1 or more and 840 or less. The foregoing means that the concentration of the second particles is at least equal to that of the first particles. When the ratio of the concentration of the second particles to the concentration of the first particles is 1 or more, the second particles may be present in a large amount with respect to the first particles. Accordingly, the depletion agglutination action of the first particles by the second particles can be satisfactorily expressed, and hence detection with higher sensitivity is achieved.

Meanwhile, when the amount of the second particles becomes excessively large with respect to that of the first particles, the viscosity of a solution containing the particles may increase to cause, for example, the following problem: air bubbles occur at the time of the fractionation of the solution or at the time of the mixing of the contents of the solution; or variation in fractionation amount of the solution occurs. In addition, the light scattering by the second particles may cause a problem in that the optical measurement of the first particles is inhibited. The ratio of the concentration of the second particles to the concentration of the first particles can be set to 840 or less because those problems affect the measurement accuracy.

From a viewpoint of the achievement of both the improvement in sensitivity and the maintenance of the measurement accuracy by the second particles, the ratio of the concentration can be 10 or more and 600 or less, or 20 or more and 250 or less.

The concentration of the second particles may be appropriately adjusted by the target substance to be measured and the material for the second particles in accordance with the average particle diameter and specific gravity thereof, and with conditions for the optical measurement. With regard to the detection reagent for a target substance of this embodiment, the concentration of the second particles in the detection reagent can be 0.08 mass % or more and 22.0 mass % or less from viewpoints of reactivity and a solution turbidity. When the concentration of the second particles is set to 0.08 mass % or more of the reagent, an effect of the second particles is clearly observed. As the amount thereof in the reagent is increased, the effect exhibited by the second particles can be improved dose-dependently.

The upper limit of the concentration of the second particles in the reagent can be at most 22.0 mass % in the reagent. This is because when the concentration falls within the range, as described above, the effect by which the sensitivity of the detection can be improved without any influence on the measurement accuracy is high. From a viewpoint of an achievement of both the improvement in sensitivity and maintenance of the measurement accuracy (reproducibility) by the second particles, the concentration of the second particles can be 0.5 or more and 20 or less, or 1.0 or more and 15.0 or less.

The second particles can be silica particles coated with PVP (hereinafter abbreviated as “PVP-coated silica particles”). The PVP shows a strong interaction with the silica particles through a hydrogen bond, and hence the PVP is not detached from the PVP-coated silica particles during the storage of the reagent or during the agglutination reaction of the first particles. The silica particles may be synthesized by an ordinary method, or a commercial product may be used. For example, the PVP-coated silica particles are obtained by synthesizing silica nanoparticles by a Stober method or a sol-gel method, and coating their surfaces with polyvinylpyrrolidone (PVP) serving as a hydrophilic polymer.

In addition, the PVP-coated silica particles are obtained by coating commercial silica nanoparticles Sicastar (manufactured by Micromod Particle Technology GmbH) with polyvinylpyrrolidone (PVP) serving as a hydrophilic polymer. In addition, the PVP-coated silica particles are suitably, for example, polyvinylpyrrolidone-coated silica particles having an average particle diameter of 20 nm, Percoll (manufactured by Cytiva). The Percoll is a solution for forming a density gradient in a solution, and is commercially available.

### (Latex Agglutination Reagent)

A latex agglutination reagent is listed as an example of the detection reagent for a target substance of this embodiment.

An example of the latex agglutination reagent of this embodiment is a latex agglutination reagent including at least: latex particles each having a site that specifically binds to the target substance; and the second particles (the particles can each be free of any site that specifically binds to the target substance). Herein, the second particles function as a sensitizer. The latex particles and second particles of this embodiment are dispersed in an aqueous solvent. Examples of the aqueous solvent include purified water and various buffers, such as a phosphate buffer, a glycine buffer, a Good buffer, a Tris buffer, an ammonia buffer, and phosphate-buffered saline. Of those, a phosphate buffer, a Good buffer, or phosphate-buffered saline (sometimes abbreviated as “PBS”) can be used.

An example of a one-liquid type latex agglutination reagent of this embodiment is described. The second particles are added to phosphate-buffered saline (sometimes abbreviated as “PBS”). In a case where the target substance is an antigen, when polystyrene particles each sensitized with an antibody that specifically binds to the target substance (sometimes termed as “antibody-sensitized latex particles”) are added to the solution, an example of the latex agglutination reagent of this embodiment can be obtained. A specimen (sometimes termed as “sample”), such as a serum or plasma, is added to a one-liquid type latex agglutination reagent, and the turbidity of the mixture is measured with a spectrophotometer.

If the specimen is free of any target substance, no change in turbidity with time is observed because the antibody-sensitized latex particles maintain a dispersed state. Meanwhile, when the specimen comprises the target substance, the antibody-sensitized latex particles agglutinate through an antigen-antibody reaction with the target substance. A change in turbidity with time is observed as a result of the agglutination. That is, the turbidity increases.

An amount of the target substance in the specimen can be determined by using a calibration curve (graph showing a relationship between the turbidity and the concentration of the target substance) obtained by turbidity measurement with a standard liquid of the target substance having a known concentration. Various additives may each be incorporated into the latex agglutination reagent of this embodiment. In general, the reagent may include, for example, a pH buffer, a protein, such as albumin or globulin, an amino acid, a surfactant, or an antiseptic.

The detection reagent for a target substance of this embodiment may include at least a first dispersion having dispersed therein the first particles and a second dispersion having dispersed therein the second particles, the dispersion being different from the first dispersion, and the respective dispersions may be independent of each other. Alternatively, the detection reagent for a target substance may include a dispersion containing at least the first particles and the second particles. Although those particle dispersions are mixed during optical measurement even in the former case, the detection reagent is regarded as including at least two solutions. A reagent including two solutions is referred to as “two-liquid type latex agglutination reagent.”

Kinds of the solutions are not limited to a specimen diluent, the first dispersion, and the second dispersion, and include, for example, a standard liquid (solution containing a known concentration of the target substance) for obtaining a calibration curve or a control for identifying measurement accuracy. A reagent set including a plurality of reagents is also referred to as “reagent kit.”

An example of the latex agglutination reagent of this embodiment is a two-liquid type latex agglutination reagent including: a first reagent (sometimes referred to as “R1 solution”) for diluting a specimen, the reagent containing the second particles; and a second reagent (sometimes referred to as “R2 solution”) containing the first particles.

Herein, the R1 solution is sometimes referred to as “specimen diluent.” In the measurement of the target substance by latex agglutination measurement with the reagent, the sensitivity of the detection (optical measurement) of the target substance can be improved by, for example, adding a specimen to the specimen diluent containing silica particles, and then mixing the solution with the R2 solution containing the antibody-sensitized latex particles.

### (Luminescent Reagent)

An example of the detection reagent for a target substance of this embodiment is a luminescent reagent including at least: luminescent particles (first particles) each having a site that specifically reacts with the target substance; and the second particles that are each free of any site that specifically reacts with the target substance.

An example of the luminescent reagent of this embodiment is described. The second particles are added to PBS. The luminescent reagent of this embodiment may be obtained by adding luminescent particles each sensitized with an antibody against the target substance (sometimes abbreviated as “antibody-sensitized luminescent particles”) to the solution. A specimen is added to the one-liquid type luminescent reagent, and the polarization degree (or polarization anisotropy) of light emitted from the antibody-sensitized luminescent particles (first particles) is measured with a fluorescence polarization-measuring apparatus.

If the specimen is free of any target substance, no change in polarization degree of the emitted light with time is observed because the antibody-sensitized luminescent particles maintain a dispersed state. Meanwhile, when the specimen comprises the target substance, the antibody-sensitized luminescent particles agglutinate through an antigen-antibody reaction with the target substance. The mobility of the antibody-sensitized luminescent particles is reduced by the agglutination.

In other words, the polarization degree of the light emitted from the antibody-sensitized luminescent particles increases. The amount of the target substance in the specimen can be determined from an increase in polarization degree by obtaining a calibration curve (graph showing a relationship between the polarization degree and the concentration of the target substance) through polarization degree measurement with a standard liquid of the target substance having a known concentration. Various additives may each be incorporated into the luminescent reagent of this embodiment.

In general, the reagent may include, for example, a pH buffer, a protein, such as albumin or globulin, an amino acid, a surfactant, or an antiseptic. As in the above-mentioned latex agglutination reagent, the luminescent reagent of this embodiment may be a reagent kit including a plurality of reagents. An example of the luminescent reagent of this embodiment is a luminescent reagent including: a specimen diluent containing silica particles; and the R2 solution containing the antibody-sensitized luminescent particles.

### Second Embodiment

A second embodiment is a method of detecting a target substance in a specimen liquid including: a first step of mixing the specimen liquid containing at least the target substance and a particle dispersion, which comprises first particles each having a site that specifically binds to the target substance, to produce a liquid sample; and a second step of performing the optical measurement of the liquid sample, wherein the liquid sample comprises second particles, wherein the ratio of the average particle diameter of the second particles to the average particle diameter of the first particles in the liquid sample is 0.03 or more and 0.24 or less, and wherein the refractive index of the second particles is smaller than the refractive index of the first particles.

Herein, the phrase “in the liquid sample” is used in a same meaning as that of the phrase “in the reaction solution.” The description of each item described in the first embodiment may be omitted in the second embodiment.

### <First Step>

The method of detecting a target substance of this embodiment is a method of detecting the target substance in the specimen liquid, and includes the first step of mixing the specimen liquid that may contain at least the target substance and the particle dispersion, which comprises the first particles each having a site that specifically binds to the target substance, to produce the liquid sample.

The first step is a step in which the first particles each having a site that specifically binds to the target substance can each specifically bind to the target substance, and the step is a step in which the first particles agglutinate depending on an amount of the target substance. In this embodiment, the agglutination of the first particles is accelerated because in this step, the second particles coexist in the liquid sample.

The sizes of the first and second particles to be used in the method of detecting a target substance of this embodiment, in other words, the average particle diameters thereof are appropriately selected in accordance with the kinds of the target substance, the detection method (a turbidity or light emission), and the particles from a viewpoint of, for example, the sensitizing effect of the particles on measurement sensitivity, or the presence or absence of hindrance on a measurement signal such as an absorbance. However, as described in the first embodiment, it is important that the average particle diameter of the second particles be smaller than that of the first particles. In addition, in the method of detecting a target substance of this embodiment, a ratio of the average particle diameter of the second particles to the average particle diameter of the first particles in the liquid sample is 0.03 or more and 0.24 or less.

As described above, when the ratio falls within the range, it is conceivable that the second particles show an effective size in the liquid sample to enable the expression of the depletion agglutination effect of the first particles, and hence the measurement sensitivity of the agglutination reaction of the particles can be improved. Meanwhile, when the ratio of the average particle diameter of the second particles to the average particle diameter of the first particles becomes excessively large, for example, when the second particles are identical in size to the first particles (the ratio between the average particle diameters is 1), a state in which the depletion agglutination occurs is no longer established, and hence the agglutination reaction of the first particles is inhibited.

In addition, when the ratio becomes excessively large, the following large demerit occurs: light scattering from the second particles significantly affects the optical measurement of the first particles; for example, measurable range of the absorbance of the liquid sample reduces.

As described above, from a viewpoint that the scattering by the second particles is wished to be reduced, sizes of the second particles can be as small as possible. Accordingly, the ratio between the average particle diameters can be 0.03 or more and 0.12 or less. From a viewpoint of the achievement of both the improvement in sensitivity and the maintenance of measurement accuracy by the second particles, the ratio between the average particle diameters can be 0.04 or more and 0.10 or less.

In addition, in the method of detecting a target substance of this embodiment, refractive index of the second particles is smaller than the refractive index of the first particles. This is because when the refractive index of the second particles is low, scattering properties from the second particles reduce, and hence no longer inhibit turbidity measurement or fluorescence polarization measurement. In the method of detecting a target substance of this embodiment, the ratio of the refractive index of the second particles to the refractive index of the first particles can be 0.92 or less. In addition, in the method of detecting a target substance of this embodiment, the refractive index of the second particles can be less than 1.59, or 1.50 or less, or 1.48 or less. When the second particles are turned into low-refractive index particles, the particles hardly affect the agglutination reaction of the first particles and the optical measurement thereof.

In addition, in the method of detecting a target substance of this embodiment, the true specific gravity of the second particles can be larger than the true specific gravity of the first particles. The foregoing is particularly useful when the first particles and the second particles are caused to coexist, and are stored for a long time period. The addition of the second particles to a reagent increases specific gravity of its solvent. As a result, the spontancous sedimentation rate of the first particles reduces, and hence dispersion stability of the first particles can be improved. The true specific gravity of the second particles can be 1.1 or more and 3.0 or less, or 1.5 or more and 2.2 or less because the second particles sediment when their specific gravity is excessively large.

As described above, the second particles can be silica particles. This is because the particles satisfy ranges of the refractive index and the specific gravity, and can be simply produced in a large amount at low cost.

In addition, in the method of detecting a target substance of this embodiment, the second particles are each free of any site that specifically binds to the target substance. This is because the agglutination reaction of the first particles is prevented from being inhibited. In this embodiment, it is important that the interaction of the second particles with a substance in a reaction solution be weakened to the extent possible.

In the method of detecting a target substance of this embodiment, the average particle diameter of the second particles can be 5 nm or more and 55 nm or less, and the average particle diameter of the first particles can be 50 nm or more and 500 nm or less. When the average particle diameters fall within the ranges, and the ranges are combined, the depletion agglutination action of the first particles by the second particles can be expressed, and hence an improvement in sensitivity of the method is achieved. In addition, the agglutination reaction of the first particles and the optical measurement thereof are hardly affected. Accordingly, the measurement range is not narrowed, and the measurement accuracy does not deteriorate.

The concentrations of the first and second particles to be used in the detection method of this embodiment are appropriately selected in accordance with the kinds of the target substance, the detection method (a turbidity or light emission), and the particles from a viewpoint of, for example, sensitizing effect of the particles on measurement sensitivity, or the presence or absence of hindrance on a measurement signal such as an absorbance. However, with regard to concentrations during the detection, it is important that the concentration of the second particles be equal to or more than that of the first particles.

In addition, in the method of detecting a target substance of the present disclosure, the concentration of the first particles in the liquid sample can be 0.0001 mass % or more and 1.0 mass % or less, and the ratio of the concentration (mass basis) of the second particles to the concentration (mass basis) of the first particles in the liquid sample can be 1 or more and 840 or less.

Under the conditions, the second particles are present in a large amount with respect to the first particles. Accordingly, the depletion agglutination action of the first particles by the second particles can be expressed, and hence an improvement in sensitivity of the method is achieved. Meanwhile, when the amount of the second particles becomes excessively large with respect to that of the first particles, the viscosity of a solution containing the particles increases to cause, for example, the following problem: air bubbles occur during the fractionation of the solution or during the mixing of the contents of the solution; or variation in fractionation amount of the solution occurs. In addition, light scattering by the second particles causes a problem in that the optical measurement of the first particles is inhibited.

A ratio of concentration of the second particles to concentration of the first particles can be set to 840 or less because those problems affect the measurement accuracy. Further, from a viewpoint of an achievement of both an improvement in sensitivity and maintenance of measurement accuracy by the second particles, the concentration ratio is 10 or more and 600 or less, or 20 or more and 250 or less.

The concentration of the second particles may be appropriately adjusted by the target substance to be measured and a material for the particles in accordance with the average particle diameter and specific gravity thereof, and with conditions for the optical measurement. In addition, in the method of detecting a target substance of this embodiment, the concentration of the second particles in the liquid sample can be 0.04 mass % or more and 11.0 mass % or less from viewpoints of reactivity and a solution turbidity. When the concentration of the second particles in the liquid sample is set to 0.04 mass % or more, an effect of the particles is clearly observed. As the amount thereof in the liquid sample is increased, the effect exhibited by the second particles can be improved dose-dependently.

An upper limit of the concentration of the second particles in the liquid sample can be at most 11.0 mass % with respect to the entire liquid sample. When the concentration falls within the range, as described above, an improving effect on the sensitivity of the method is obtained without any influence on the measurement accuracy. Further, from the viewpoint of the achievement of both the improvement in sensitivity and the maintenance of the measurement accuracy by the second particles, the concentration of the second particles in the liquid sample is 0.3 mass % or more and 10.0 mass % or less, or 0.5 mass % or more and 7.5 mass % or less.

In the method of detecting a target substance of this embodiment, the second particles can serve as a sensitizer in the detection of the target substance and as a dispersant for the first particles. With such setting, there can be performed a detection method including using a detection reagent, which has satisfactory storage stability and hence can be stored for a long time period, can be performed.

### <Second Step>

The method of detecting a target substance of this embodiment includes the second step of performing the optical measurement of the liquid sample. The method of detecting a target substance in a specimen of this embodiment includes the step of performing the optical measurement of the liquid sample produced in accordance with the foregoing. The target substance is detected by examining an extent of the agglutination of the first particles through the optical measurement of the liquid sample. The target substance may be detected qualitatively or quantitatively. In the detection, the formation of an aggregate may be visually observed. However, from viewpoints of the reproducibility and throughput of the detection, an extent of the particle agglutination can be measured with an optical measuring apparatus.

In the method of detecting a target substance in a specimen of this embodiment, a relational equation (calibration curve) between the concentration of the target substance and the extent of the particle agglutination may be determined in advance by using, for example, a solution of the target substance having a known concentration. The concentration of the target substance may be determined from the extent of the particle agglutination obtained by measuring the target substance. Herein, the extent of the particle agglutination may be measured by the absorbance or fluorescence polarization degree of the liquid sample to be optically measured.

(Example in which Target Substance is Detected by Immunoassay)

The detection method of this embodiment is specifically described below by taking an immunoassay method as an example. The term “immunoassay method” as used herein refers to an immunoassay method based on an antigen-antibody reaction, and includes an immunoassay method called, for example, latex agglutination immunonephelometry, a latex agglutination method, or a fluorescence polarization immunoassay method. In the method of detecting a target substance of this embodiment, the optical measurement uses a latex agglutination method or a fluorescence polarization immunoassay method.

### (Latex Agglutination Method Including Using Latex Particles)

In the latex agglutination method in this embodiment, the target substance may be qualitatively and quantitatively measured by: adding the specimen containing the target substance to a latex agglutination reagent including latex particles (antibody-sensitized latex particles) each having bonded thereto an antibody against the target substance and the second particles that are each free of any site that specifically binds to the target substance; and visually or optically detecting the agglutination of the aggregate of the latex particles produced by an antigen-antibody reaction. From viewpoints of sensitivity and the avoidance of absorption derived from contaminants in the specimen, a detection wavelength can fall within a visible range, and can be a wavelength of 400 nm or more and 800 nm or less.

As an example, the specimen containing the target substance (antigen) is mixed into a particle dispersion containing antibody-sensitized latex particles and silica particles to produce a liquid sample. Next, the absorbance of the liquid sample is measured. At this time, the ratio of the average particle diameter of the silica particles to the average particle diameter of the antibody-sensitized latex particles is 0.03 or more and 0.24 or less, and the refractive index of the silica particles is smaller than the refractive index of the antibody-sensitized latex particles.

Under the conditions, high-sensitivity latex agglutination measurement can be performed. The reason why the conditions are used is as described above. This is because a depletion agglutination action by the silica particles is efficiently expressed, and the silica particles do not optically affect a change in absorbance derived from the antibody-sensitized latex particles.

The ratio of the concentration of the silica particles to the concentration of the antibody-sensitized latex particles in the liquid sample is particularly 1 or more and 230 or less. A latex agglutination reaction can be performed while the concentration of the silica particles is set within the range of from 0.04 mass % or more to 11.0 mass % or less. The setting may be appropriately performed so that an amount of the specimen, an amount of the first reagent, and an amount of the second reagent may satisfy the above-mentioned ranges. With such setting, during the latex agglutination reaction, viscosities of the respective solutions for forming the reagents are low, and hence the solutions are easy to handle. Accordingly, the accuracy of the measurement is improved.

In the detection of the target substance by the latex agglutination method, the concentration of the antibody-sensitized latex particles in the liquid sample particularly falls within the range of, for example, from 0.005 mass % or more to 1.9 mass % or less. In the range, the change in absorbance of the antibody-sensitized latex particles can be accurately detected.

The latex agglutination method is generally performed at a reaction temperature in the range of from 4° C. or more to 50° C. or less. When the temperature is low, antigen-antibody reactivity reduces, and when the temperature is high, the stability of an antigen-antibody immune complex reduces. Accordingly, the temperature can be 10° C. or more and 40° C. or less, or 30° C. or more and 40° C. or less. The latex agglutination method is generally performed for a reaction time in a range of 0 minutes or more to 60 minutes or less. The reaction time can fall within the range of from 1 minute or more to 30 minutes or less.

### (Fluorescence Polarization Immunoassay Method Including Using Luminescent Particles)

In the fluorescence polarization immunoassay method in this embodiment, the target substance may be qualitatively and quantitatively measured by: adding the specimen that may contain the target substance to a luminescent reagent including luminescent particles (antibody-sensitized luminescent particles) each having bonded thereto an antibody against the target substance and the second particles that are each free of any site that specifically binds to the target substance; and detecting the agglutination of the aggregate of the luminescent particles, which has been produced by an antigen-antibody reaction, by a fluorescence polarization method. From viewpoints of sensitivity and the avoidance of absorption derived from contaminants in the specimen, the wavelength at which light emitted from the reagent is detected can fall within a visible range, and can be a wavelength of 400 nm or more and 800 nm or less.

The fluorescence polarization method has been used for analyzing the mobility of a fluorescent molecule in a solution. The principle of the fluorescence polarization method is described for the case of the luminescent particles of this embodiment. When the luminescent particles in a liquid (luminescent molecules showing fluorescence polarization properties are incorporated into the particles) are excited by plane-polarized light, the particles each emit polarized fluorescence toward one and the same plane. However, when the luminescent particles are rotated by Brownian motion while being in an excited state, their fluorescence polarization is canceled because the particles each emit fluorescence toward a plane different from the excitation plane. In other words, the term “fluorescence polarization degree” refers to the degree of the rotational motion of the luminescent particles during a period from their excitation to the emission of the fluorescence.

When the luminescent particles in the liquid are monodispersed in the solution, the particles show a low polarization degree because the particles are vigorously rotated by their Brownian motion. Meanwhile, when the luminescent particles agglutinate, their Brownian motion in the solution reduces, and hence the polarization degree increases. Accordingly, in the fluorescence polarization method, the mobility of the luminescent particles in the solution is analyzed by using a change in polarization degree as an indicator. A milli P (hereinafter abbreviated as “mp”) representing a change in plane-polarized light may be used as an indicator of the polarization degree. In the fluorescence polarization immunoassay method in this embodiment, the agglutination reaction of the antibody-sensitized luminescent particles, which occurs depending on an antigen amount, may be evaluated by the polarization degree mp.

A mode of the detection of the target substance by the fluorescence polarization immunoassay method is same as the latex agglutination method including using the latex particles described above. However, the concentration of the antibody-sensitized luminescent particles can fall within the range of from 0.0001 mass % or more to 0.009 mass % or less because the sensitivity of the fluorescence measurement is high. In the range, the fluorescence intensity of the antibody-sensitized luminescent particles and a change in fluorescence polarization degree thereof can be accurately detected. Although the fluorescence polarization method is also referred to as “fluorescence depolarization method,” the terms “fluorescence polarization method” and “fluorescence depolarization method” as used herein are identical in meaning to each other.

(Method of Storing First Particles Each Having Site that Specifically Binds to Target Substance)

This embodiment includes a method of storing the first particles each having a site that specifically binds to the target substance in a reagent including the second particles that are each free of any site that specifically binds to the target substance. The term “storage” as used herein means that the particles are stored at a predetermined temperature for a predetermined time period. A storage method is as follows: the above-mentioned reagent of the present disclosure, which includes the first particles each having a site that specifically binds to the target substance and the second particles that are each free of any site that specifically binds to the target substance, only needs to be stored.

Although the storage temperature is not particularly limited, for example, the temperature can be 0° C. or more and 50° C. or less, or 0° C. or more and 20° C. or less, or 0° C. or more and 10° C. or less. In such range, the effect of the second particles is obtained. Although the storage period is not particularly limited, the period is, for example, 2 days, 7 days, 14 days, 1 month, 3 months, 6 months, or 1 year, and only needs to be selected in accordance with the storage temperature and the usage method of the reagent. As the amount of the second particles is increased, the suppressing effect of the second particles on the spontaneous sedimentation of the first particles is expressed dose-dependently. Accordingly, the target substance-detecting performance of the first particles can be stably maintained.

### (Others)

In the method of detecting a target substance of this embodiment, the first particles can be polystyrene-containing particles (the particles may be polystyrene particles), and the second particles can be silica-containing particles (the particles may be silica particles).

In the foregoing description, the first step and second step of the method of detecting a target substance of this embodiment seem to be steps to be performed at separate timings. However, the first step and the second step may be similarly performed.

### EXAMPLES

Some examples (hereinafter referred to as “Examples”) for more specifically describing the present disclosure are described below. However, the present disclosure is not limited thereto.

### Example 1: Preparation of Latex Agglutination Reagent for Detecting CRP

A latex agglutination reagent formed of the following particles was prepared: latex particles each having a site that specifically bound to a target substance were used as first particles; and silica particles that were each free of any site that specifically bound to the target substance were used as second particles.

Percoll was used as the silica particles. The Percoll is silica particles whose surfaces are each coated with PVP, and their analysis by a dynamic light scattering method recognized that the particles had an average particle diameter (2-average particle diameter) of 20 nm. The silica particle concentration of the Percoll was 23 mass %.

Antibody-sensitized latex particles were prepared in accordance with a known method as described below.

Polystyrene latex particles (particles having a z-average particle diameter of 213.5 nm: manufactured by Fujikura Kasci Co., Ltd.) were used as particles. 50 Microliters of a solution of the polystyrene latex particles having a particle concentration of 10% and 950 μL of a 10 mM HEPES buffer (pH: 7.0) were loaded into a microtube to prepare a latex particle dispersion. A water-soluble carbodiimide (WSC) was dissolved in a HEPES buffer (pH: 7.0) (WSC concentration: 1 mg/mL). 300 Microliters of the WSC solution was added to the latex particle dispersion (1 mL). The mixture in the microtube was stirred with a tube mixer at room temperature for 30 minutes.

Next, 0.3 mg of an anti-human CRP polyclonal antibody (manufactured by Oriental Yeast Co., Ltd.) was loaded into the microtube, and the mixture was stirred at room temperature for 1 hour. The mixture was centrifuged at 4° C. and 20,000 G for 10 minutes, and the supernatant was removed. After that, the residue was washed with an albumin-containing HEPES buffer twice, and was redispersed with a HEPES buffer. The resultant antibody-sensitized polystyrene particles are hereinafter referred to as “antibody-sensitized latex particles LA1.” An analysis of the antibody-sensitized latex particles LA1 by a dynamic light scattering method recognized that the particles had an average particle diameter (z-average particle diameter) of 238.3 nm.

Percoll having a silica particle concentration of 23 mass % and the antibody-sensitized latex particles LA1 were mixed into a HEPES buffer to prepare a reagent S1 in which the concentration of the antibody-sensitized latex particles LA1 was 0.025 mass % and the concentration of the silica particles was 0.092 mass % (Tables 1-1 to 1-6).

The following contents for the reagent S1 are shown in Tables 1-1 to 1-6: the average particle diameters of the particles (the average particle diameters are z-average particle diameters obtained by a dynamic light scattering method, and the average particle diameter of the first particles and the average particle diameter of the second particles are shown as d1 and d2, respectively); a ratio (c2/c1) of the concentration of the second particles to the concentration of the first particles; a ratio (d2/d1) of the z-average particle diameter of the second particles to the z-average particle diameter of the first particles; materials for the particles; the refractive indices of the particles; the specific gravities of the particles; and coating materials for the surfaces of the particles. In Tables 1-1 to 1-6, the c1, the c2, a c1′, and a c2′ are each shown as a value adjusted to 3 decimal places by rounding the fourth decimal place, and the ratios “c2/c1” and “c2′/c1” are each shown as a value adjusted to 3 decimal places as follows: the ratio “c2/c1” or “c2′/c1” is calculated without rounding the calculated c1, c2, c1′, and c2′, and the fourth decimal place of the value thus calculated is rounded.

Refractive indices of the particles were determined by using the Lorentz-Lorenz equation. A refractive index of the polystyrene particles was 1.59, and a refractive index of the silica particles was 1.45. Accordingly, a ratio of the refractive index of the second particles whose material is silica to that of the first particles whose material is polystyrene is 0.92, though the value is not shown in Tables 1-1 to 1-6.

The specific gravity of the polystyrene particles and the specific gravity of the silica particles obtained by a dry density measurement method were 1.05 and 1.8, respectively.

The first particles of the reagent S1 were evaluated for their stability. In the stability evaluation, an absorbance change ratio on a 14th day was used as an indicator. Specifically, absorbances of the reagent immediately after its preparation and after its refrigerated storage for 14 days were measured (measurement wavelength: 572 nm, optical path length: 1 cm), and a ratio of an absorbance after the refrigerated storage for 14 days to an absorbance immediately after the preparation (i.e., the change ratio) was determined.

When the antibody-sensitized latex particles LA1 can be stably dispersed by the refrigerated storage for 14 days, the change ratio is 100% because absorbance does not change. When the antibody-sensitized latex particles LA1 are instable, for example, when the particles spontaneously sediment, the absorbance reduces with time, and hence the ratio also reduces. It was found that stability of the reagent S1 was 90.3%, and was hence higher than that of a comparative reagent C1 to be described later, that is, 85%, (i.e., it can be said that measurement accuracy is high in the long run). In addition, from such viewpoint, the second particles may be used as a sensitizer and a dispersant.

The reagent S1 was evaluated for its measurable range. In terms of reagent performance, the measurable range can be as large as possible because the concentration of the target substance can be measured in a wide range. In the measurable range evaluation, to the reagent S1 immediately after its preparation, a HEPES buffer whose amount was equal to that of the reagent was added, and the absorbance of the resultant solution was measured (measurement wavelength: 572 nm, optical path length: 1 cm). Herein, a measurement upper limit was set to 2.0 because the measurement upper limit of an absorbance in spectrometry was about 2.0.

Accordingly, the measurable range was represented as a value obtained by subtracting absorbance of the reagent S1 from the upper limit absorbance, and the upper limit absorbance is, 2.0. For example, absorbance of the reagent S1 was 0.5, and hence the measurable range of the reagent S1 was 1.5 (value obtained by subtracting 0.5 from 2.0). As described later, the value was comparable to that of the comparative reagent C1 which is free of silica particles, and hence no influences of the silica particles on the measurable range were observed.

In addition, another viewpoint of the measurable range evaluation is the inhibition of the silica particles on a change in absorbance of the antibody-sensitized latex particles LA1. In other words, it is the antibody-sensitized latex particles LA1 that react with the target substance to change its absorbance, and hence ideally, an absorbance derived from the silica particles is desirably as small as possible. This is because an absorbance derived from the antibody-sensitized latex particles LA1 in the reagent and the absorbance derived from the silica particles cannot be distinguished from each other.

The percentage by which the absorbance derived from the antibody-sensitized latex particles LA1 is increased by adding the silica particles can be 20% or less. This is because of the following reason: when the absorbance is increased by more than the percentage, as described above, the measurable range narrows, and the possibility that a change in absorbance derived from the antibody-sensitized latex particles LA1 cannot be accurately observed becomes higher.

### Example 2: Preparation Example of Latex Agglutination Reagent

A reagent S2 was prepared in a same manner as in Example 1 except that concentration of the silica particles was 0.69 mass %.

The respective parameters for the reagent S2 are shown in Tables 1-1 to 1-6. An evaluation was performed in the same manner as in Example 1. As a result, it was found that stability of the reagent S2 was 92.8%, and was hence higher than 85%, which is a stability of the comparative reagent C1 to be described later.

The measurable range of the reagent S2 was 1.5. No influences of the silica particles on the measurable range were observed.

### Example 3: Preparation Example of Latex Agglutination Reagent

A reagent S3 was prepared in a same manner as in Example 1 except that the concentration of the silica particles was 1.38 mass %.

The respective parameters for the reagent S3 are shown in Tables 1-1 to 1-6. An evaluation was performed in a same manner as in Example 1. As a result, it was found that the stability of the reagent S3 was 96.8%, and was hence higher than 85%, which is a stability of the comparative reagent C1 to be described later.

The measurable range of the reagent S3 was 1.5. No influences of the silica particles on the measurable range were observed.

### Example 4: Preparation Example of Latex Agglutination Reagent

A reagent S4 was prepared in a same manner as in Example 1 except that the concentration of the silica particles was 5.52 mass %.

The respective parameters for the reagent S4 are shown in Tables 1-1 to 1-6. An evaluation was performed in a same manner as in Example 1. As a result, it was found that the stability of the reagent S4 was 98.3%, and was hence higher than 85%, which is a stability of the comparative reagent C1 to be described later.

The measurable range of the reagent S4 was 1.5. No influences of the silica particles on the measurable range were observed.

### Example 5: Preparation Example of Latex Agglutination Reagent

A reagent S5 was prepared in a same manner as in Example 1 except that the concentration of the silica particles was 11.5 mass %.

The respective parameters for the reagent S5 are shown in Tables 1-1 to 1-6. An evaluation was performed in a same manner as in Example 1. As a result, it was found that the stability of the reagent S5 was 99.7%, and was hence higher than 85%, which is a stability of the comparative reagent C1 to be described later.

The measurable range of the reagent S5 was 1.5. No influences of the silica particles on the measurable range were observed.

### Example 6: Preparation Example of Latex Agglutination Reagent

A reagent S6 was prepared in a same manner as in Example 1 except that the concentration of the silica particles was 16.1 mass %.

The respective parameters for the reagent S6 are shown in Tables 1-1 to 1-6. An evaluation was performed in a same manner as in Example 1. As a result, it was found that the stability of the reagent S6 was 99.1%, and was hence higher than 85%, which is a stability of the comparative reagent C1 to be described later.

The measurable range of the reagent S6 was 1.4. Almost no influences of the silica particles on the measurable range were observed.

### Example 7: Preparation Example of Latex Agglutination Reagent

A reagent S7 was prepared in a same manner as in Example 1 except that the concentration of the silica particles was 20.7 mass %.

The respective parameters for the reagent S7 are shown in Tables 1-1 to 1-6. An evaluation was performed in the same manner as in Example 1. As a result, it was found that the stability of the reagent S7 was 99.2%, and was hence higher than 85%, which is a stability of the comparative reagent C1 to be described later.

The measurable range of the reagent S7 was 1.4. Almost no influences of the silica particles on the measurable range were observed.

### Example 8: Preparation Example of Latex Agglutination Reagent

Commercial silica nanoparticles Sicastar (manufactured by Micromod Particle Technology GmbH, refractive index: 1.45) were each coated with polyvinylpyrrolidone (PVP) serving as a hydrophilic polymer. First, to an aqueous solution (particle concentration: 2.5%) of the Sicastar having an average particle diameter (z-average particle diameter) of 10 nm, an aqueous solution (PVP concentration: 1%) of polyvinylpyrrolidone (PVP-K30: manufactured by Tokyo Chemical Industry Co., Ltd.) whose amount was equal to that of the foregoing solution was added.

After that, the Sicastar coated with PVP was separated by ultrafiltration, and was washed with water to provide the Sicastar coated with PVP. A reagent S8 was prepared in a same manner as in Example 1 except that: the PVP-coated silica particles were used; and the concentration of the PVP-coated silica particles was 5.52 mass % (Tables 1-1 to 1-6).

The respective parameters for the reagent S8 are shown in Tables 1-1 to 1-6. An evaluation was performed in a same manner as in Example 1. As a result, it was found that the stability of the reagent S8 was 92.1%, and was hence higher than 85%, which is a stability of the comparative reagent C1 to be described later.

The measurable range of the reagent S8 was 1.5. No influences of the silica particles on the measurable range were observed.

### Example 9: Preparation Example of Latex Agglutination Reagent

A reagent S9 was prepared in a same manner as in Example 8 except that the average particle diameter of the silica particles was 30 nm. The respective parameters for the reagent S9 are shown in Tables 1-1 to 1-6. An evaluation was performed in a same manner as in Example 1. As a result, it was found that the stability of the reagent S9 was 96.7%, and was hence higher than 85%, which is a stability of the comparative reagent C1 to be described later.

The measurable range of the reagent S9 was 1.5. No influences of the silica particles on the measurable range were observed.

### Example 10: Preparation Example of Latex Agglutination Reagent

A reagent S10 was prepared in a same manner as in Example 8 except that the average particle diameter of the silica particles was 50 nm. The respective parameters for the reagent S10 are shown in Tables 1-1 to 1-6. An evaluation was performed in the same manner as in Example 1. As a result, it was found that the stability of the reagent S10 was 95.5%, and was hence higher than the stability of the comparative reagent C1 to be described later, that is, 85%.

The measurable range of the reagent S10 was 1.4. Almost no influences of the silica particles on the measurable range were observed.

### Comparative Example 1

The comparative reagent C1 was prepared in a same manner as in Example 1 except that the silica particles were not incorporated (the concentration of the second particles was 0%). The respective parameters for the comparative reagent C1 are shown in Tables 1-1 to 1-6.

An evaluation was performed in a same manner as in Example 1. As a result, it was found that the stability of the comparative reagent C1 was 85.0%, and was hence lower than those of the reagents of Examples of the present disclosure. This is probably because the antibody-sensitized latex particles LA1 spontaneously sediment with time. The measurable range of the comparative reagent C1 was 1.5.

### Comparative Example 2

A comparative reagent C2 was prepared in a same manner as in Example 8 except that the average particle diameter of the silica particles was 70 nm. The respective parameters for the comparative reagent C2 are shown in Tables 1-1 to 1-6. An evaluation was performed in a same manner as in Example 1. As a result, the measurable range of the comparative reagent C2 was 1.2. The measurable range narrowed because the absorbance of the silica particles was higher than those of the reagents of Examples. In addition, in the comparative reagent C2, the absorbance derived from the antibody-sensitized latex particles LA1 and the absorbance derived from the silica particles were not able to be distinguished from each other. Accordingly, the stability of the comparative reagent C2 was not evaluated because its measurement using a change in absorbance of the antibody-sensitized latex particles LA1 as an indicator was not able to be performed.

### Comparative Example 3

A comparative reagent C3 was prepared in a same manner as in Example 8 except that the average particle diameter of the silica particles was 100 nm. The respective parameters for the comparative reagent C3 are shown in Tables 1-1 to 1-6. An evaluation was performed in a same manner as in Example 1. As a result, the measurable range of the comparative reagent C3 was 0.1. The measurable range narrowed because the absorbance of the silica particles was higher than those of the reagents of Examples. In addition, in the comparative reagent C3, the absorbance derived from the antibody-sensitized latex particles LA1 and the absorbance derived from the silica particles were not able to be distinguished from each other. Accordingly, the stability of the comparative reagent C3 was not evaluated because its measurement using a change in absorbance of the antibody-sensitized latex particles LA1 as an indicator was not able to be performed.

### Example 11: Latex Agglutination Method Assay

The latex agglutination reagents of Examples were each evaluated for its sensitivity with C-reactive protein (hereinafter abbreviated as “CRP”). In the assay, 1 μL of a CRP solution having a CRP concentration of 0.5 mg/dL was added to 50 μL of phosphate-buffered saline (first reagent). The mixture was loaded into the measurement cell (optical path length: 10 mm) of a spectrophotometer, and was held at 37° C. for 5 minutes.

Next, 50 μL of the reagent S1 serving as the second reagent of Examples was added to 51 μL of the above-mentioned first reagent, and the reagents were sufficiently mixed. After that, the absorbance of the mixture at 572 nm was measured with the spectrophotometer (the absorbance is defined as an absorbance at 0 minutes). 5 Minutes thereafter, the absorbance thereof at 572 nm was measured again (the absorbance is defined as an absorbance after 5 minutes). In Tables 1-1 to 1-6, the concentration of the first particles in the mixed liquid (mixed liquid of 50 μL of the reagent S1 and 51 μL of the first reagent), which had been subjected to the above-mentioned absorbance measurement, was represented by c1′, the concentration of the second particles therein was represented by c2′, and the ratio “c2′/c1” was calculated.

A difference ((absorbance after 5 minutes)−(absorbance at 0 minutes)) between the resultant two absorbances is determined, and a value obtained by multiplying the difference by 10,000 is represented as an absorbance change amount (AOD×10,000). When particle agglutination is caused by an antibody-antigen reaction between the first particles and CRP, the absorbance change amount becomes higher. A higher absorbance change amount means higher sensitivity. The average of values obtained by performing the measurement in three times was determined as the absorbance change amount.

The sensitizing factor of the CRP measurement of the reagent S1 of Example 1 was evaluated in accordance with the above-mentioned measurement method. The sensitizing factor of the CRP measurement was determined by setting the absorbance change amount of Comparative Example 4 to be described later to 100%.

The coefficient of variation (CV) of the absorbance change amount of the CRP measurement of the reagent S1 of Example 1 was determined in accordance with the above-mentioned measurement method. A smaller coefficient of variation means higher measurement accuracy (i.e., higher reproducibility).

The reagent S1 was examined for its absorbance change amount in accordance with a same measurement method as that described above except that in the above-mentioned measurement method, the CRP concentration was set to 0, in other words, 1 μL of phosphate-buffered saline was added to 50 μL of the first reagent. This is a test for examining a latex agglutination reaction for whether or not nonspecific agglutination occurs. When the absorbance change amount was a value close to zero (100 or less), it was judged that no nonspecific agglutination occurred, and when the absorbance change amount was more than 100, it was judged that the nonspecific agglutination occurred.

The results are shown in Tables 1-1 to 1-6. As shown in Tables 1-1 to 1-6, in the reagent S1 (the concentration of the silica particles during the absorbance measurement was 0.046%) in which the antibody-sensitized latex particles LA1 and the silica particles were mixed, the reagent serving as the second reagent, the sensitizing factor of CRP measurement was 121%. The foregoing means that the sensitivity of the CRP measurement is improved as compared to that of Comparative Example 4 to be described later, which is free of any silica particles. Accordingly, it was recognized that the addition of the silica particles to the reagent had a sensitivity-improving effect. The coefficient of variation of the CRP measurement was 8.2%, and was hence comparable to that of Comparative Example 4. The addition of the silica particles to the reagent showed no change in coefficient of variation. In the reagent S1, no nonspecific agglutination occurred.

### Example 12: Latex Agglutination Method Assay

The reagent S2 in which the concentration of the silica particles during its absorbance measurement was 0.342% was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S2 was 179%. The sensitivity of the CRP measurement was improved as compared to that of Comparative Example 4 to be described later, which was free of any silica particles. The coefficient of variation of the CRP measurement was 7.8%, and was hence comparable to that of Comparative Example 4. In the reagent S2, no nonspecific agglutination occurred.

### Example 13: Latex Agglutination Method Assay

The reagent S3 in which the concentration of the silica particles during its absorbance measurement was 0.683% was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S3 was 242%. The sensitivity of the CRP measurement was improved as compared to that of Comparative Example 4 to be described later, which was free of any silica particles. The coefficient of variation of the CRP measurement was 8.6%, and was hence comparable to that of Comparative Example 4. In the reagent S3, no nonspecific agglutination occurred.

### Example 14: Latex Agglutination Method Assay

The reagent S4 in which the concentration of the silica particles during its absorbance measurement was 2.733% was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S4 was 263%. The sensitivity of the CRP measurement was improved as compared to that of Comparative Example 4 to be described later, which was free of any silica particles. The coefficient of variation of the CRP measurement was 7.9%, and was hence comparable to that of Comparative Example 4. In the reagent S4, no nonspecific agglutination occurred.

### Example 15: Latex Agglutination Method Assay

The reagent S5 in which the concentration of the silica particles during its absorbance measurement was 5.693% was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S5 was 256%. The sensitivity of the CRP measurement was improved as compared to that of Comparative Example 4 to be described later, which was free of any silica particles. The coefficient of variation of the CRP measurement was 9.7%, and was hence comparable to that of Comparative Example 4. In the reagent S5, no nonspecific agglutination occurred.

### Example 16: Latex Agglutination Method Assay

The reagent S6 in which the concentration of the silica particles during its absorbance measurement was 7.97% was evaluated for the sensitizing factor and coefficient of variation of its CRP measurement, and nonspecific agglutination in a same manner as in Example 11 (Tables 1-1 to 1-6).

The sensitizing factor of the CRP measurement of the reagent S6 was 285%. The sensitivity of the CRP measurement was improved as compared to that of Comparative Example 4 to be described later, which was free of any silica particles. Although the coefficient of variation of the CRP measurement was 12.0%, and was hence larger than that of Comparative Example 4, the coefficient of variation fell within an allowable range (20% or less). In the reagent S6, no nonspecific agglutination occurred.

### Example 17: Latex Agglutination Method Assay

The reagent S7 in which the concentration of the silica particles during its absorbance measurement was 10.248% was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S7 was 279%. The sensitivity of the CRP measurement was improved as compared to that of Comparative Example 4 to be described later, which was free of any silica particles. Although the coefficient of variation of the CRP measurement was 18.8%, and was hence larger than that of Comparative Example 4, the coefficient of variation fell within an allowable range (20% or less). In the reagent S7, no nonspecific agglutination occurred.

### Example 18: Latex Agglutination Method Assay

The reagent S8, in which the concentration of the silica particles during its absorbance measurement was 2.733% and the average particle diameter of the silica particles was 10 nm, was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S8 was 181%. The sensitivity of the CRP measurement was improved as compared to that of Comparative Example 4 to be described later, which was free of any silica particles. Although the coefficient of variation of the CRP measurement was 11.3%, and was hence larger than that of Comparative Example 4, the coefficient of variation fell within an allowable range (20% or less). In the reagent S8, no nonspecific agglutination occurred.

### Example 19: Latex Agglutination Method Assay

The reagent S9, in which the concentration of the silica particles during its absorbance measurement was 2.733% and the average particle diameter of the silica particles was 30 nm, was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S9 was 136%. The sensitivity of the CRP measurement was improved as compared to that of Comparative Example 4 to be described later, which was free of any silica particles. Although the coefficient of variation of the CRP measurement was 12.7%, and was hence larger than that of Comparative Example 4, the coefficient of variation fell within an allowable range (20% or less). In the reagent S9, no nonspecific agglutination occurred.

### Example 20: Latex Agglutination Method Assay

The reagent S10, in which the concentration of the silica particles during its absorbance measurement was 2.733% and the average particle diameter of the silica particles was 50 nm, was evaluated for the sensitizing factor and coefficient of variation of its CRP measurement, and nonspecific agglutination in a same manner as in Example 11 (Tables 1-1 to 1-6). The sensitizing factor of the CRP measurement of the reagent S10 was 112%. The sensitivity of the CRP measurement was improved as compared to that of Comparative Example 4 to be described later, which was free of any silica particles. Although the coefficient of variation of the CRP measurement was 15.9%, and was hence larger than that of Comparative Example 4, the coefficient of variation fell within an allowable range (20% or less). In the reagent S10, no nonspecific agglutination occurred.

### Comparative Example 4

The comparative reagent C1 that was free of any silica particles during its absorbance measurement (the concentration of the silica particles was 0%) was evaluated for the sensitizing factor and coefficient of variation of its CRP measurement, and nonspecific agglutination in a same manner as in Example 11 (Tables 1-1 to 1-6). The sensitizing factor of the CRP measurement of the comparative reagent C1 was set to 100%. The coefficient of variation of the CRP measurement was 8.2%. In the comparative reagent C1, no nonspecific agglutination occurred.

### Comparative Example 5

The comparative reagent C2, in which the concentration of the silica particles during its absorbance measurement was 2.733% and the average particle diameter of the silica particles was 70 nm, was evaluated for the sensitizing factor and coefficient of variation of its CRP measurement, and nonspecific agglutination in a same manner as in Example 11 (Tables 1-1 to 1-6). The sensitizing factor of the CRP measurement of the comparative reagent C2 was 81%, and was hence lower than that of Comparative Example 4 that was free of any silica particles. The coefficient of variation of the CRP measurement was as large as 22.4%. In the comparative reagent C2, the nonspecific agglutination was observed. Those results may be due to the fact that the average particle diameter of the silica particles serving as the second particles is large. In other words, the results may be caused by the fact that the addition of the large silica particles inhibits the agglutination reaction of the first particles, and the fact that the absorbance derived from the silica particles reduces measurement accuracy.

### Comparative Example 6

The comparative reagent C3, in which the concentration of the silica particles during its absorbance measurement was 2.733% and the average particle diameter of the silica particles was 100 nm, was evaluated for the sensitizing factor and coefficient of variation of its CRP measurement, and nonspecific agglutination in a same manner as in Example 11 (Tables 1-1 to 1-6). The sensitizing factor of the CRP measurement of the comparative reagent C3 was 55%, and was hence lower than that of Comparative Example 4 that was free of any silica particles. The coefficient of variation of the CRP measurement was as large as 28.7%. In the comparative reagent C2, the nonspecific agglutination was observed. Those results may be due to the fact that the average particle diameter of the silica particles serving as the second particles is large. In other words, the results may be caused by the fact that the addition of the large silica particles inhibits the agglutination reaction of the first particles, and the fact that the absorbance derived from the silica particles reduces measurement accuracy.

TABLE 1-1

| | | | Example | | | | |
|---|---|---|---|---|---|---|---|
| | | | 1 | 2 | 3 | 4 | 5 |
| Reagent including first particles and second particles | | | S1 | S2 | S3 | S4 | S5 |
| Particle | Concentration of first | c1 | 0.025 | 0.025 | 0.025 | 0.025 | 0.025 |
| concentration | particles each having site | | | | | | |
| | that specifically reacts with | | | | | | |
| | target substance | | | | | | |
| | (mass %) | | | | | | |
| | Concentration of second | c2 | 0.092 | 0.69 | 1.38 | 5.52 | 11.5 |
| | particles that are each free | | | | | | |
| | of site that specifically | | | | | | |
| | reacts with target substance | | | | | | |
| | (mass %) | | | | | | |
| | Ratio of concentration of | c2/c1 | 3.68 | 27.6 | 55.2 | 220.8 | 460 |
| | second particles to | | | | | | |
| | concentration of first | | | | | | |
| | particles | | | | | | |
| Average | z-Average particle diameter | d1 | 238 | 238 | 238 | 238 | 238 |
| particle | of first particles | | | | | | |
| diameter | (nm) | | | | | | |
| | z-Average particle diameter | d2 | 20 | 20 | 20 | 20 | 20 |
| | of second particles | | | | | | |
| | (nm) | | | | | | |
| | Ratio of z-average particle | d2/d1 | 0.084 | 0.084 | 0.084 | 0.084 | 0.084 |
| | diameter of second particles | | | | | | |
| | to z-average particle | | | | | | |
| | diameter of first particles | | | | | | |
| Material | Material for first particles | | Polystyrene | Polystyrene | Polystyrene | Polystyrene | Polystyrene |
| | Material for second | | Silica | Silica | Silica | Silica | Silica |
| | particles | | | | | | |
| Refractive | Refractive index of first | | 1.59 | 1.59 | 1.59 | 1.59 | 1.59 |
| index of | particles | | | | | | |
| material | Refractive index of second | | 1.45 | 1.45 | 1.45 | 1.45 | 1.45 |
| | particles | | | | | | |
| Specific | True specific gravity of first | | 1.05 | 1.05 | 1.05 | 1.05 | 1.05 |
| gravity of | particles | | | | | | |
| particles | True specific gravity of | | 1.8 | 1.8 | 1.8 | 1.8 | 1.8 |
| | second particles | | | | | | |
| Surface | Coating material for | | Antibody | Antibody | Antibody | Antibody | Antibody |
| coating | surfaces of first particles | | | | | | |
| material | Coating material for | | PVP | PVP | PVP | PVP | PVP |
| | surfaces of second particles | | | | | | |
| Stability | Absorbance change ratio on | | 90.3 | 92.8 | 96.8 | 98.3 | 99.7 |
| evaluation | 14th day | | | | | | |
| | (ΔOD 14/ΔOD 0) % | | | | | | |
| Measurable | Measurable range of absorbance | | 1.5 | 1.5 | 1.5 | 1.5 | 1.5 |
| range | (measurement wavelength: | | | | | | |
| evaluation | 572 nm) | | | | | | |

TABLE 1-2

| | | | Example | | | | |
|---|---|---|---|---|---|---|---|
| | | | 6 | 7 | 8 | 9 | 10 |
| Reagent including first particles and second particles | | | S6 | S7 | S8 | S9 | S10 |
| Particle | Concentration of first particles | c1 | 0.025 | 0.025 | 0.025 | 0.025 | 0.025 |
| concentration | each having site that specifically | | | | | | |
| | reacts with target substance | | | | | | |
| | (mass %) | | | | | | |
| | Concentration of second particles | c2 | 16.1 | 20.7 | 5.52 | 5.52 | 5.52 |
| | that are each free of site | | | | | | |
| | that specifically reacts with | | | | | | |
| | target substance | | | | | | |
| | (mass %) | | | | | | |
| | Ratio of concentration of second | c2/c1 | 644 | 828 | 220.8 | 220.8 | 220.8 |
| | particles to concentration of first | | | | | | |
| | particles | | | | | | |
| Average | z-Average particle diameter of | d1 | 238 | 238 | 238 | 238 | 238 |
| particle | first particles | | | | | | |
| diameter | (nm) | | | | | | |
| | z-Average particle diameter of | d2 | 20 | 20 | 10 | 30 | 50 |
| | second particles | | | | | | |
| | (nm) | | | | | | |
| | Ratio of z-average particle | d2/d1 | 0.084 | 0.084 | 0.042 | 0.126 | 0.210 |
| | diameter of second particles to z- | | | | | | |
| | average particle diameter of first | | | | | | |
| | particles | | | | | | |
| Material | Material for first particles | | Polystyrene | Polystyrene | Polystyrene | Polystyrene | Polystyrene |
| | Material for second particles | | Silica | Silica | Silica | Silica | Silica |
| Refractive | Refractive index of first particles | | 1.59 | 1.59 | 1.59 | 1.59 | 1.59 |
| index of | Refractive index of second | | 1.45 | 1.45 | 1.45 | 1.45 | 1.45 |
| material | particles | | | | | | |
| Specific | True specific gravity of first | | 1.05 | 1.05 | 1.05 | 1.05 | 1.05 |
| gravity of | particles | | | | | | |
| particles | True specific gravity of second | | 1.8 | 1.8 | 1.8 | 1.8 | 1.8 |
| | particles | | | | | | |
| Surface | Coating material for surfaces of | | Antibody | Antibody | Antibody | Antibody | Antibody |
| coating | first particles | | | | | | |
| material | Coating material for surfaces of | | PVP | PVP | PVP | PVP | PVP |
| | second particles | | | | | | |
| Stability | Absorbance change ratio on | | 99.1 | 99.2 | 92.1 | 96.7 | 95.5 |
| evaluation | 14th day | | | | | | |
| | (ΔOD 14/ΔOD 0) % | | | | | | |
| Measurable | Measurable range of absorbance | | 1.4 | 1.4 | 1.5 | 1.5 | 1.4 |
| range | (measurement wavelength: | | | | | | |
| evaluation | 572 nm) | | | | | | |

TABLE 1-3

| | | | Example | | | | |
|---|---|---|---|---|---|---|---|
| | | | 11 | 12 | 13 | 14 | 15 |
| Particle | Concentration of first | c1′ | 0.012 | 0.012 | 0.012 | 0.012 | 0.012 |
| concentration | particles each having site | | | | | | |
| | that specifically reacts with | | | | | | |
| | target substance during | | | | | | |
| | optical measurement | | | | | | |
| | (mass %) | | | | | | |
| | Concentration of second | c2′ | 0.046 | 0.342 | 0.683 | 2.733 | 5.693 |
| | particles that are each free of | | | | | | |
| | site that specifically reacts | | | | | | |
| | with target substance during | | | | | | |
| | optical measurement | | | | | | |
| | (mass %) | | | | | | |
| | Ratio of concentration of | c2′/c1′ | 3.68 | 27.6 | 55.2 | 220.8 | 460 |
| | second particles to | | | | | | |
| | concentration of first | | | | | | |
| | particles during optical | | | | | | |
| | measurement | | | | | | |
| Average | Ratio of z-average particle | d2/d1 | 0.084 | 0.084 | 0.084 | 0.084 | 0.084 |
| particle | diameter of second particles | | | | | | |
| diameter | to z-average particle | | | | | | |
| | diameter of first particles | | | | | | |
| Sensitivity | Absorbance change amount | ΔOD × 10,000 | 580 | 860 | 1,160 | 1,260 | 1,230 |
| Evaluation | when CRP concentration is | | | | | | |
| | 0.5 mg/dL | | | | | | |
| | (ΔOD × 10,000) | | | | | | |
| | Sensitizing factor when CRP | | 121% | 179% | 242% | 263% | 256% |
| | concentration is 0.5 mg/dL % | | | | | | |
| | (with respect to | | | | | | |
| | Comparative Example 4) | | | | | | |
| Reproducibility | Coefficient of variation | CV | 8.2% | 7.8% | 8.6% | 7.9% | 9.7% |
| evaluation | when CRP concentration is | | | | | | |
| | 0.5 mg/dL | | | | | | |
| | (reproducibility) % | | | | | | |
| Nonspecific | Absorbance change amount | ΔOD × 10,000 | −130 | −70 | −10 | −50 | −80 |
| agglutination | when CRP concentration is | | | | | | |
| evaluation | 0 mg/dL | | | | | | |
| | (ΔOD × 10,000) | | | | | | |

TABLE 1-4

| | | | Example | | | | |
|---|---|---|---|---|---|---|---|
| | | | 16 | 17 | 18 | 19 | 20 |
| Particle | Concentration of first | c1′ | 0.012 | 0.012 | 0.012 | 0.012 | 0.012 |
| concentration | particles each having site | | | | | | |
| | that specifically reacts with | | | | | | |
| | target substance during | | | | | | |
| | optical measurement | | | | | | |
| | (mass %) | | | | | | |
| | Concentration of second | c2′ | 7.970 | 10.248 | 2.733 | 2.733 | 2.733 |
| | particles that are each free of | | | | | | |
| | site that specifically reacts | | | | | | |
| | with target substance during | | | | | | |
| | optical measurement | | | | | | |
| | (mass %) | | | | | | |
| | Ratio of concentration of | c2′/c1′ | 644 | 828 | 220.8 | 220.8 | 220.8 |
| | second particles to | | | | | | |
| | concentration of first | | | | | | |
| | particles during optical | | | | | | |
| | measurement | | | | | | |
| Average | Ratio of z-average particle | d2/d1 | 0.084 | 0.084 | 0.042 | 0.126 | 0.210 |
| particle | diameter of second | | | | | | |
| diameter | particles to z-average | | | | | | |
| | particle diameter of first | | | | | | |
| | particles | | | | | | |
| Sensitivity | Absorbance change amount | ΔOD × 10,000 | 1,370 | 1,340 | 870 | 655 | 539 |
| Evaluation | when CRP concentration is | | | | | | |
| | 0.5 mg/dL | | | | | | |
| | (ΔOD × 10,000) | | | | | | |
| | Sensitizing factor when | | 285% | 279% | 181% | 136% | 112% |
| | CRP concentration is 0.5 | | | | | | |
| | mg/dL % | | | | | | |
| | (with respect to | | | | | | |
| | Comparative Example 4) | | | | | | |
| Reproducibility | Coefficient of variation | CV | 12.0% | 18.8% | 11.3% | 12.7% | 15.9% |
| evaluation | when CRP concentration is | | | | | | |
| | 0.5 mg/dL | | | | | | |
| | (reproducibility) % | | | | | | |
| Nonspecific | Absorbance change amount | ΔOD × 10,000 | −80 | −90 | 30 | 70 | 100 |
| agglutination | when CRP concentration is | | | | | | |
| evaluation | 0 mg/dL | | | | | | |
| | (ΔOD × 10,000) | | | | | | |

TABLE 1-5

| | | | Comparative Example | | |
|---|---|---|---|---|---|
| | | | 1 | 2 | 3 |
| Reagent including first particles and second particles | | | C1 | C2 | C3 |
| Particle | Concentration of first particles | c1 | 0.025 | 0.025 | 0.025 |
| concentration | each having site that specifically | | | | |
| | reacts with target substance (mass %) | | | | |
| | Concentration of second particles that | c2 | 0 | 5.52 | 5.52 |
| | are each free of site that specifically | | | | |
| | reacts with target substance (mass %) | | | | |
| | Ratio of concentration of second | c2/c1 | 0 | 220.8 | 220.8 |
| | particles to concentration of first | | | | |
| | particles | | | | |
| Average | z-Average particle diameter of first | d1 | 238 | 238 | 238 |
| particle | particles (nm) | | | | |
| diameter | z-Average particle diameter of second | d2 | — | 70 | 100 |
| | particles (nm) | | | | |
| | Ratio of z-average particle diameter of | d2/d1 | — | 0.294 | 0.420 |
| | second particles to z-average particle | | | | |
| | diameter of first particles | | | | |
| Material | Material for first particles | | Polystyrene | Polystyrene | Polystyrene |
| | Material for second particles | | — | Silica | Silica |
| Refractive | Refractive index of first particles | | 1.59 | 1.59 | 1.59 |
| index of | Refractive index of second particles | | — | 1.45 | 1.45 |
| material | | | | | |
| Specific | True specific gravity of first particles | | 1.05 | 1.05 | 1.05 |
| gravity of | True specific gravity of second particles | | — | 1.8 | 1.8 |
| particles | | | | | |
| Surface | Coating material for surfaces of first | | Antibody | Antibody | Antibody |
| coating | particles | | | | |
| material | Coating material for surfaces of second | | — | PVP | PVP |
| | particles | | | | |
| Stability | Absorbance change ratio on 14th day | | 85 | Not | Not |
| evaluation | (ΔOD 14/ΔOD 0) % | | | evaluated | evaluated |
| Measurable | Measurable range of absorbance | | 1.5 | 1.2 | 0.1 |
| range | (measurement wavelength: 572 nm) | | | | |
| evaluation | | | | | |

TABLE 1-6

| | | | Comparative Example | | |
|---|---|---|---|---|---|
| | | | 4 | 5 | 6 |
| Particle | Concentration of first particles each | c1′ | 0.012 | 0.012 | 0.012 |
| concentration | having site that specifically reacts | | | | |
| | with target substance during optical | | | | |
| | measurement (mass %) | | | | |
| | Concentration of second particles that | c2′ | 0 | 2.733 | 2.733 |
| | are each free of site that specifically | | | | |
| | reacts with target substance during | | | | |
| | optical measurement (mass %) | | | | |
| | Ratio of concentration of second | c2′/c1′ | 0 | 220.8 | 220.8 |
| | particles to concentration of first | | | | |
| | particles during optical measurement | | | | |
| Average | Ratio of z-average particle diameter of | d2/d1 | — | 0.294 | 0.420 |
| particle | second particles to z-average particle | | | | |
| diameter | diameter of first particles | | | | |
| Sensitivity | Absorbance change amount when CRP | ΔOD × | 480 | 387 | 264 |
| evaluation | concentration is 0.5 mg/dL | 10,000 | | | |
| | (ΔOD × 10,000) | | | | |
| | Sensitizing factor when CRP | | 100% | 81% | 55% |
| | concentration is 0.5 mg/dL % (with | | | | |
| | respect to Comparative Example 4) | | | | |
| Reproducibility | Coefficient of variation when CRP | CV | 8.2% | 22.4% | 28.7% |
| evaluation | concentration is 0.5 mg/dL | | | | |
| | (reproducibility) % | | | | |
| Nonspecific | Absorbance change amount when CRP | ΔOD × | 50 | 157 | 323 |
| agglutination | concentration is 0 mg/dL | 10,000 | | | |
| evaluation | (ΔOD × 10,000) | | | | |

According to the present disclosure, there can be provided a method of detecting a target substance and a detection reagent therefor that each have high sensitivity and high accuracy.

Further, according to the present disclosure, there can also be provided a detection reagent for a target substance that has satisfactory storage stability and hence can be stored for a long time period.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-152067, filed Sep. 4, 2024, which is hereby incorporated by reference herein in its entirety.

What is claimed is:

1. A method of detecting a target substance comprising:
a first step of preparing a liquid sample by mixing a specimen liquid that may comprise the target substance with a particle dispersion, which particle dispersion comprises first particles each having a site that specifically binds to the target substance; and
a second step of performing optical measurement of the liquid sample,
wherein the liquid sample additionally comprises second particles,
wherein a ratio of an average particle diameter of the second particles to an average particle diameter of the first particles in the liquid sample is 0.03 or more and 0.24 or less, and
wherein a refractive index of the second particles is smaller than a refractive index of the first particles.

2. The method of detecting a target substance according to claim 1, wherein a ratio of the refractive index of the second particles to the refractive index of the first particles is 0.92 or less.

3. The method of detecting a target substance according to claim 1, wherein the refractive index of the second particles is 1.50 or less.

4. The method of detecting a target substance according to claim 1, wherein a true specific gravity of the second particles is larger than a true specific gravity of the first particles.

5. The method of detecting a target substance according to claim 1, wherein the average particle diameter of the second particles is 5 nm or more and 55 nm or less.

6. The method of detecting a target substance according to claim 1, wherein a concentration of the second particles in the liquid sample is 0.04 mass % or more and 11.0 mass % or less.

7. The method of detecting a target substance according to claim 1, wherein a ratio of a concentration (mass basis) of the second particles to a concentration (mass basis) of the first particles in the liquid sample is 1 or more and 840 or less.

8. The method of detecting a target substance according to claim 1, wherein a concentration of the first particles in the liquid sample is 0.0001 mass % or more and 1.0 mass % or less.

9. The method of detecting a target substance according to claim 1, wherein the optical measurement uses one of a latex agglutination method or a fluorescence polarization immunoassay method.

10. A detection reagent for a target substance comprising at least:
first particles each having a site that specifically binds to the target substance; and
second particles that are different in kind from the first particles,
wherein a ratio of an average particle diameter of the second particles to an average particle diameter of the first particles is 0.03 or more and 0.24 or less, and
wherein a refractive index of the second particles is smaller than a refractive index of the first particles.

11. The detection reagent for a target substance according to claim 10, wherein a ratio of the refractive index of the second particles to the refractive index of the first particles is 0.92 or less.

12. The detection reagent for a target substance according to claim 10, wherein the refractive index of the second particles is 1.50 or less.

13. The detection reagent for a target substance according to claim 10, wherein a true specific gravity of the second particles is larger than a true specific gravity of the first particles.

14. The detection reagent for a target substance according to claim 10, wherein the average particle diameter of the second particles is 5 nm or more and 55 nm or less.

15. The detection reagent for a target substance according to claim 10, wherein a concentration of the second particles in the detection reagent is 0.08 mass % or more and 22.0 mass % or less.

16. The detection reagent for a target substance according to claim 10,
wherein the first particles are particles each containing polystyrene, and
wherein the second particles are silica particles.

17. The detection reagent for a target substance according to claim 10, wherein the average particle diameter of the first particles is 50 nm or more and 500 nm or less.

18. The detection reagent for a target substance according to claim 10, wherein a ratio of a concentration (mass basis) of the second particles to a concentration (mass basis) of the first particles in the detection reagent is 1 or more and 840 or less.

19. The detection reagent for a target substance according to claim 10, wherein a concentration of the first particles in the detection reagent is 0.0002 mass % or more and 2.0 mass % or less.

20. The detection reagent for a target substance according to claim 10, wherein the target substance is detected by using one of a latex agglutination method or a fluorescence polarization immunoassay method.
